(19) United States
(12) Patent Application Publication
(10) Pub. No.: US 2023/0190928 A1
(43) Pub. Date: Jun. 22, 2023

(54) CONTROLLED RELEASE VACCINE FORMULATION

(71) Applicant: Phibro Animal Health Corporation, Teaneck, NJ (US)

(72) Inventors: Avner Finger, Pardes Hanna-Karkur (IL); Yevgeny Khinich, Petah Tikva (IL)

(73) Assignee: Phibro Animal Health Corporation, Teaneck, NJ (US)

(21) Appl. No.: 18/154,304

(22) Filed: Jan. 13, 2023

Related U.S. Application Data
(63) Continuation of application No. PCT/IB21/56318, filed on Jul. 14, 2021.
(60) Provisional application No. 63/054,056, filed on Jul. 20, 2020.

(51) Int. Cl. A61K 39/39; A61K 9/107; A61P 37/04

(52) U.S. Cl. CPC A61K 39/39; A61K 9/107; A61P 37/04; A61K 2039/55511

(57) ABSTRACT

Vaccine formulations comprising polyglycerol polyricinoleate (PGPR) are disclosed. Certain disclosed exemplary vaccine formulations comprised an aqueous phase comprising inactivated bacteria and/or viruses, and/or bacterial and/or viral antigens. One particular embodiment comprised an inactivated H9N2 PGPR emulsion-based vaccine for day-old chicks. Disclosed PGPR-based vaccine formulations can be administered alone, or in combination with or as a composition including a second standard fast release vaccine. Disclosed vaccines delay antigen release, and therefore delay an immune response in a subject receiving the vaccine, typically by 7-35 days. The present invention also concerns a method for vaccinating a subject, such as poultry or fish, with disclosed vaccine formulations, as well as a method for making PGPR-based vaccine formulations.

### CROSS REFERENCE TO RELATED APPLICATION

The present application is a continuation of International Application No. PCT/M2021/056318, filed on Jul. 14, 2021, which was published in English under PCT Article 21(2), which in turn claims the benefit of the earlier filing date of U.S. Provisional Application No. 63/054,056, filed on Jul. 20, 2020, both of which prior applications are incorporated herein by reference in their entireties.

### BACKGROUND

Commercial production of food-producing animals requires protecting animals against large numbers of different pathogens. Inactivated and live vaccines are used either in combination or separately to build and boost the immunity and protect the animal. Inactivated vaccine formulations are based on adjuvanted antigen(s). Inactivated vaccine compositions may be formulated in a variety of different forms, such as water-in-oil, oil-in-water, water-in-oil-in-water emulsions, and others.

Immunizing animals with vaccines requires restraining the animal and injecting the vaccine into the animal's muscle or into the sub-cutaneous tissue, which causes the animal stress. Recently, the industry is trying to reduce the number of injections in order to reduce animal stress and suffering. One approach that has been developed to ostensibly reduce the number of injections is using combined vaccines that include several antigens in the same dose. Most of these vaccines require a second booster injection in order to boost the immune response and specific IgG/IgY production. This concept of priming and boosting is one of the key fundamentals in immunology, and repeated vaccination is a common practice that requires two or more injections for each animal with an interval of a few weeks between injections.

One of the factors that affects immunization efficacy in young, such as day-old chicks, is the Maternal Derived Antibody (MDA) level. A high MDA delays or may even block the immune response against the homologous antigen.

### SUMMARY

Certain disclosed embodiments of the present invention concern a new vaccine formulation. The disclosed formulation provides several benefits, including allowing priming and boosting of the animal's immune system using a single vaccine injection. Certain disclosed embodiments also present a new system for controlled and delayed antigen release in vivo that addresses issues associated with MDA reducing or entirely blocking the immune response in offspring.

One disclosed embodiment of the present invention concerns a first vaccine formulation comprising polyglycerol polyricinoleate (PGPR), such as 0.01% to 6% PGPR, more typically 0.05% to 2.0% PGPR, and even more typically 0.6% to 1.5%, by volume. A person of ordinary skill in the art will appreciate that embodiments of applicant's PGPR vaccine formulations can be used with any suitable antigens including, by way of example and without limitation, bacterial and/or viral antigens selected from: inactivated Newcastle Disease Virus (NDV) and/or at least one NDV immunogenic protein or portion thereof, such as a recombinant or naturally occurring HN and/or F protein or subunit(s) thereof; inactivated Infectious Bursal Disease Virus (IBDV) and/or at least one IBDV immunogenic protein or portion thereof; inactivated Avian Influenza Virus (AI) and/or at least one AI immunogenic protein or portion thereof; inactivated Infectious Bronchitis Virus (IBV) and/or at least one IBV immunogenic protein or portion thereof; inactivated Avian Reo Virus (ARV) and/or at least one ARV immunogenic protein or portion thereof; inactivated Avian Metapneumovirus (AMPV) and/or at least one AMPV immunogenic protein or portion thereof; inactivated Avian Metapneumovirus (AMPV) and/or at least one AMPV immunogenic protein or portion thereof; inactivated Salmonella and/or an inactivated Salmonella immunogenic protein or portion thereof; inactivated Avibacterium paragallinarum and/or an inactivated Avibacterium paragalhnarum immunogenic protein or portion thereof; inactivated Escherichia coli and/or an inactivated Escherichia coli immunogenic protein or portion thereof; and/or inactivated Pasteurella multocida and/or an inactivated immunogenic protein or portion thereof.

The first PGPR vaccine formulation may be used in combination with one or more additional vaccines. For example, a controlled-release PGPR vaccine according to the present invention may be used either in combination with, or as a single composition comprising, a second standard fast release vaccine. The standard fast release vaccine may include any inactivated virus or bacteria, and/or viral or bacterial antigens suitable for treating animals, including but not limited to those expressly identified herein for administration using PGPR emulsion vaccines. The PGPR vaccine may be an inactivated, emulsion vaccine composition that includes a first micelle based on PGPR comprising emulsion entrained antigens, and a second micelle, such as may be formed using a polysorbate, comprising the same or different emulsion entrained antigens.

PGPR delays antigen release. PGPR also provides a controlled, slow release once antigen release is initiated. As a result, an immune response is delayed in a subject receiving the PGPR-based vaccine formulation. This delay in an immune response is generally at least 7 days, and is more typically 21-35 days, after the PGPR vaccine is administered, relative to a subject receiving a substantially identical vaccine except that it does not include PGPR. This allows antigens to be released in the subject receiving the PGPR-based vaccine formulation when the MDA level does not preclude an immune response.

One particular example of a vaccine formulation according to the present invention comprised an inactivated Avian Influenza H9N2 PGPR emulsion-based vaccine for day-old chicks.

One particular exemplary embodiment comprised 21% of an aqueous emulsion phase comprising 20% H9N2 inactivated antigen, and 1% Polysorbate 80; and 79% of an oil emulsion phase comprising 76% mineral oil, 3% Sorbitan oleate, and 0.1-1.25% PGPR.

The present invention also concerns a combination comprising a first inactivated emulsion vaccine formulation comprising polyglycerol polyricinoleate (PGPR), and a second standard fast release vaccine. The vaccines may be administered separately in any order or simultaneously. The combination may be a composition comprising the first and second vaccines, as discussed above.

The present invention also provides a method for vaccinating a subject with a disclosed PGPR-based vaccine combination or vaccine composition according to the present disclosure. Certain exemplary trials involved vaccinating day-old broiler chicks with 0.2 ml /dose to 0.3 ml/dose of a PGPR-base vaccine formulation. For an H9N2 PGPR emulsion-based vaccine, such method resulted in H9N2 induced HI titers higher than 3 at 28 and 35 days of age, and 0.1 and 0.75% PGPR formulations comprising inactivated H9N2 induced titers higher than 4 in about 60% of the birds with an HI of 3. The emulsion vaccine comprising PGPR induced active production of antibodies against the inactivated H9N2 when administered to day-old chicks despite maternal antibodies. The method may further comprise vaccinating the subject with a PGPR-based vaccine formulation according to the present invention in combination with a standard vaccine that does not comprise PGPR, whereby the standard vaccine induces a priming effect, and the PGPR formulation induces a boost effect a few weeks later. The method may involve a mixing a PGPR emulsion together with a standard vaccine (fast release), either at the formulation stage or just prior to injection to the subject.

A method for making an inactivated emulsion vaccine comprising PGPR also is disclosed. The method may involve forming an aqueous phase comprising inactivated antigens; forming an oil phase comprising PGPR; and forming an emulsion comprising the aqueous and oil phases. An emulsion can be formed using PGPR by itself, such as by using about 2% PGPR by volume. Alternatively, the emulsion can be formed using PGPR in combination with a surfactant or surfactants, such as polysorbate 80 and/or sorbitan oleate.

### BRIEF DESCRIPTION OF THE DRAWINGS

FIG. 1 provides antibody titer results, mortality rates, and percent survival for day-old chicks vaccinated with a commercial vaccine and an inactivated PGPR emulsion-based Newcastle disease vaccine according to the present invention.

FIG. 2 is a graph of titer versus time subsequent to an intramuscular administration of an 0.3 ml dose of a PGPR-based vaccine formulation and administration of an 0.3 ml dose of a commercial vaccination that compares titer production as a result of administering the two different vaccine formulations.

FIG. 3 is a graph of titer versus time subsequent to an intramuscular administration of an 0.012 ml dose of a PGPR-based vaccine formulation and administration of an 0.012 ml dose of a commercial vaccination that compares titer production as a result of administering the two different vaccine formulations.

FIG. 4 is a schematic drawing illustrating antibody production (μg ml⁻¹ serum) following administration of a vaccine A at time zero followed by administration of vaccine A+B at a time subsequent to time zero, illustrating the lag phase, the response to vaccine A, and the response to vaccine A+B.

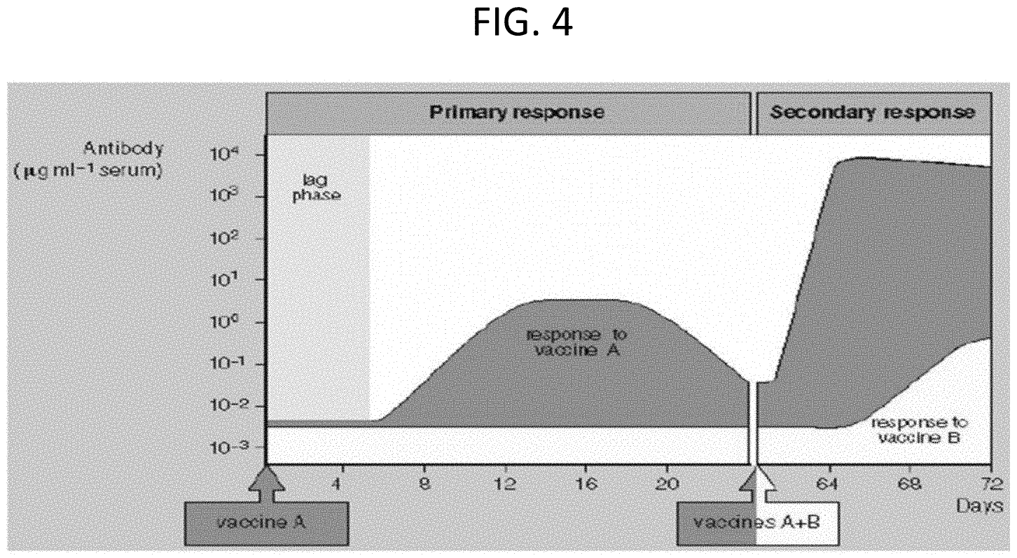

FIG. 5 is graph of antibody levels versus days post vaccination for trials described in Example 1.

### DETAILED DESCRIPTION

### I. TERMS AND ABBREVIATIONS

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Combination: A combination comprises two or more components that are administered such that the effective time period of the first component overlaps with the effective time period of the second and subsequent components. A combination may be a composition comprising the components, such that the components are administered simultaneously, or a combination may be two or more separate components that are administered substantially simultaneously, or sequentially in any order.

Emulsion: A stable mixture of two or more immiscible substances wherein one substance (i.e., the disperse phase or minor component) is dispersed within the other (i.e., the continuous phase or major component). For example, cream is an emulsion in which water surrounds droplets of oil, i.e., an oil-in-water emulsion. An emulsifier is a substance that aids in forming and maintaining an emulsion. Common emulsifiers include proteins, carbohydrate polymers, and long-chain alcohols and fatty acids, among others.

Excipient: A substance that is substantially physiologically inert and that is used as an additive in a pharmaceutical composition. An excipient can be used, for example, to dilute an active agent and/or to modify properties of a pharmaceutical composition.

Pharmaceutically acceptable: A substance that can be taken into a subject without significant adverse toxicological effects on the subject. The term “pharmaceutically acceptable form” means any pharmaceutically acceptable derivative or variation, such as stereoisomers, stereoisomer mixtures, enantiomers, solvates, hydrates, isomorphs, polymorphs, pseudomorphs, neutral forms, salt forms, and prodrug agents.

Pharmaceutically acceptable carrier: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, Pa., 21st Edition (2005), describes compositions and formulations suitable for pharmaceutical delivery of one or more therapeutic compositions and additional pharmaceutical agents. In general, the nature of the carrier will depend on the particular mode of administration being employed. For instance, disclosed vaccine formulations may comprise pharmaceutically and physiologically acceptable injectable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain other non-toxic auxiliary substances, such as wetting or emulsifying agents, preservatives, such as gentamycin, penicillin, and/or thimerosal, pH buffering agents and the like, for example sodium acetate or sorbitan monolaurate. In some examples, the pharmaceutically acceptable carrier is a non-naturally occurring or synthetic carrier. The carrier also can be formulated in a unit-dosage form that carries a preselected therapeutic dosage of the active agent, for example in a syringe.

Pharmaceutically acceptable salt: A biologically compatible salt of a compound that can be used as a drug, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, benzene sulfonic acid (besylate), cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19, which is incorporated herein by reference.)

Subject: An animal (human or non-human) subjected to a treatment, observation or experiment. “Subject” includes, by way of example and without limitation, both human and veterinary subjects, such as human and non-human mammals, mice, cats, dogs, pigs, poultry, horses, ruminants, bovines, cows, non-human primates, and aquatic species, such as fish.

Surfactant/surface active material: A compound that reduces surface tension when dissolved in water or water solutions, or that reduces interfacial tension between two liquids. A surfactant molecule typically has a polar or ionic “head” and a nonpolar hydrocarbon “tail.” Upon dissolution in water, the surfactant molecules aggregate and form micelles, in which the nonpolar tails are oriented inward and the polar or ionic heads are oriented outward toward the aqueous environment. Micelles typically are spherical in shape and small, with diameters of less than about 10 nm. The nonpolar tails create a nonpolar “pocket” within the micelle. Nonpolar compounds in the solution are sequestered in the pockets formed by the surfactant molecules, thus allowing the nonpolar compounds to remain mixed within the aqueous solution.

Therapeutically effective amount or dose: An amount sufficient to provide a beneficial, or therapeutic, effect to a subject or a given percentage of subjects.

Therapeutic time window: The length of time during which an effective dose, or therapeutic dose, of a compound remains therapeutically effective in vivo.

Treating or treatment: With respect to disease, either term includes (1) preventing the disease, e.g., causing the clinical symptoms of the disease not to develop in an animal that may be exposed to or predisposed to the disease but does not yet experience or display symptoms of the disease, (2) inhibiting the disease, e.g., arresting the development of the disease or its clinical symptoms, or (3) relieving the disease, e.g., causing regression of the disease or its clinical symptoms.

%: All percentages stated herein are percentages based on volume, unless expressly noted otherwise.

### II. EMULSION FORMULATION TYPES

Certain disclosed embodiments involve forming emulsions for delivery of antigens, such as inactivated bacteria and/or viruses, and/or bacterial antigen, viral antigens, or combinations thereof. An emulsion is a dispersion of a liquid, called the dispersed phase, in a second liquid, called the continuous phase, with which the first liquid is not miscible. For vaccine formulations, these phases typically are aqueous (antigenic media) and oil. Surfactants may be added in order to stabilize the emulsions. Surfactants can be defined by their Hydrophilic:Lipohilic Balance (HLB) value, which provides information concerning their relative affinity for both phases. Different types of emulsions can be made based on the HLB value of the surfactant. Those having a low HLB value have a high affinity for oily phases and render water-in-oil (W/O) emulsions, where antigenic phase droplets disperse into a continuous oily phase. Surfactants having a high HLB value have a high affinity for the aqueous phase and render oil-in-water (O/W) emulsions, where the continuous phase is water and the dispersed phase is oil. And finally, with certain surfactant systems having an intermediate HLB value, water-in-oil-in-water (W/O/W) emulsions can be made where the continuous phase is aqueous, the dispersed phase is oil, but the oil droplets also entrap an aqueous phase.

Different mode of action mechanisms are involved for different vaccine emulsions and their adjuvant effect. A first mechanism is, for example, a depot effect, which provides slow antigen release from the injection site. The depot effect is not the only mechanism. For example, microdiffusion of oil droplets to the draining lymph nodes can partly explain how the adjuvant effect is maintained after the emulsion is excised from the injection site.

Kinetic antigen release by an emulsion varies according to the emulsion type too. Whereas protein without adjuvant is immediately released, O/W emulsions allow a slight delay, but protein is still released relatively quickly. Alternatively, W/O emulsions allow no or slower antigen release, which correlates with the stability of the emulsion. As soon as the emulsion breaks down, large amounts of antigen are released, but slower than O/W emulsions. W/O/W emulsions have an in intermediate antigen release behavior.

Emulsions also protect antigen from rapid physiological degradation, such as in vivo degradation by enzymes, and can modify the electric charge of the antigen to become immunogenic. Emulsions also may create an inflammatory response and stimulate the recruitment of antigen-presenting cells such as macrophages and lymphocytes. They are also able to favor the uptake of antigens by antigen presenting cells (APC).

### 1. Water-in-Oil Emulsions

Water-in-oil emulsions are recommended for bovine, small ruminants, poultry and fish when long term immunity is required. For foot and mouth disease, for example, mineral oil-based emulsions can protect bovines for 1 year with one vaccination, whereas formulations based on aluminum hydroxide require two boosts or more.

Water-in-oil emulsions allow the vaccine dose or the antigen concentration to be reduced. This is an important consideration for making vaccines cost effective.

Certain embodiments of the present invention concern water-in-oil emulsions. A person of ordinary skill in the art will appreciate that the relative amounts of water and oil in the base water-in-oil composition can vary such as, by way of example, from 20% to 35% aqueous phase, typically about 30% water, and 65% to 80% oil phase, typically about 70% oil. The oil fraction can be any oil suitable for forming water-in-oil vaccine emulsions, such as mineral oil, for example a C₁₅-C₃₀ mineral oil, a vegetable oil, a synthetic oil, or combinations thereof.

### 2. Water-in-Oil-in-Water Emulsion

Water-in-oil-in-water Emulsion (W/O/W) emulsions are characterized by low viscosity and their ability to enhance short- and long-term immune response. The antigen in the external aqueous phase is immediately available to the immune system like aqueous formulations, whereas antigen in the internal aqueous phase is slowly released, like water-in-oil emulsions.

3. Oil-in-water Emulsions

Oil in water emulsions are very fluid, well tolerated and induce strong, short-term immune responses. The oil phase ratio is very low, between 15 and 25%, which partly explains their safety.

### III. POLYGLYCEROL POLYRICINOLEATE (PGPR) AND VACCINE FORMULATIONS COMPRISING PGPR

### A. PGPR

Disclosed formulations according to the present invention comprise polyglycerol polyricinoleate (PGPR), which is made from polymerized glycerol and polymerized ricinoleic acid. Ricinoleic acid [preferred IUPAC name=(9Z,12R)-12-hydroxyoctadec-9-enoic acid, but is also known as 12-hydroxy-9-cis-octadecenoic acid], shown below, is an unsaturated, omega-9 fatty acid that occurs naturally in mature castor plant (Ricinus communis L. Euphorbiacea) seeds.

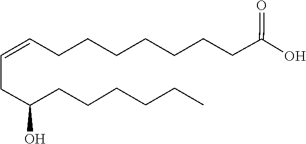 [SMILES: CCCCCC[C@@H](O)C/C=C\CCCCCCCC(=O)O]

### Ricinoleic Acid

Polyglycerol polyricinoleate (PGPR) is understood to have the following structural formula

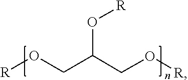

where R is

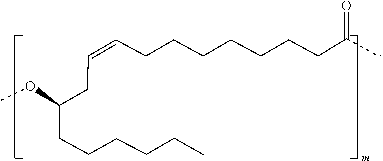

With reference to these formulas, n, the average degree of polymerization of the glycerol unit, is 5, and m, the average condensation number of ricinoleic acid, is 2.

PGPR is known to be safe for ingestion or administration. PGPR is, for example, widely known as an excellent water-in-oil emulsifier in the food industry because it forms very stable emulsions even when the water content is very high, such as 80%. PGPR is used as an emulsifier in tin-greasing emulsions for the baking industry. However, the main PGPR application is in the chocolate industry. PGPR has been used continuously in greasing emulsions since 1952, following short-term rat feeding trials undertaken in 1951, and was first used in chocolate couverture in the UK in 1952. Accordingly, the safety of PGPR consumption has been widely studied.

The PGPR safety testing program in the 1950s included acute toxicity studies in several species, 30-and 45-week rat feeding trials, a rat reproduction study over three generations, and a number of indirect metabolism studies. These studies established that PGPR is digested and utilized like a normal dietary fat. In the 1960s, the program was extended to include 2-year rat and 80-week mouse feeding studies, a 90-day feeding study in a nonrodent (chicken), studies of PGPR-induced liver and kidney enlargement in rats, mice and chickens, and rat metabolism using radio-labelled materials. Human studies also were conducted to determine the digestibility and absorption of PGPR, which included both liver and kidney function tests. PGPR was found to be 98% digested by rats and utilized as a source of energy superior to starch and nearly equivalent to groundnut oil. There was no interference with normal fat metabolism in rats or in the utilization of fat-soluble vitamins. Despite the intimate relationship with fat metabolism, no evidence was found of any adverse effects on such vital processes as growth, reproduction, and maintenance of tissue homeostasis. PGPR was not carcinogenic in either 2-year rat or 80-week mouse feeding studies.

In 1969, the 13th report of the Joint FAO/WHO Expert Committee on Food Additives (JECFA) set a temporary human acceptable daily intake (ADI) for PGPR of 3.75 mg/kg body weight with a request for more biological studies. This ADI, without the temporary prefix, was raised to 7.5 mg/kg body weight in the 17th report of JECFA in 1974. In 1979, the Scientific Committee for Food (SCF) of the European Community also set an ADI for PGPR of 7.5 mg/kg body weight. The maximum PGPR use levels in foodstuffs ready for consumption in Europe are listed below in Table 1.

TABLE 1
Maximum levels of PGPR in foodstuff,
European Directive 95/2/EC

| | | | Maximum |
|---|---|---|---|
| : Number | Name | Foodstuff | Level |
| | | Spreadable fats as defined in | 4 g/kg |
| | | Annexes A, B, and C of | |
| | | Regulation (EC) Number | |
| | | 2991/94 having a fat content of | |
| | | 41% or less | |
| :−476 | Polyglycerol | Similar spreadable products | 4 g/kg |
| | polyricinoleate | with a fat content of less than | |
| | | 10% fat | |
| | | Dressings | 4 g/kg |
| | | Cocoa-based confectionery, | 5 g/kg |
| | | including chocolate | |

Formulations according to the present invention typically comprise 0.015% to 6% PGPR, more typically 0.05% to 5% PGPR, such as 0.06% to 1.5% PGPR or 0.1% to 1.25% PGPR.

### B. Diseases Treated and Immunogenic Components Therefor

Disclosed vaccine embodiments comprising PGPR can be used to treat a variety of viral and bacterial diseases. Accordingly, disclosed vaccine formulations comprise PGPR and a bacterial and/or viral immunogenic component. For fowl, such as chickens, and aquaculture, such as fish, such diseases include, by way of example and without limitation:

Newcastle Disease Virus (NDV), provided as an inactivated virus, and/or as an NDV immunogenic protein or portion thereof, such as a recombinant or naturally occurring HN protein, F protein, subunit(s) thereof, and combinations thereof. A typical NDV antigen concentration is 50 PD₅₀/dose (50% protective dose), or stated alternatively, 5-50 μg/dose;

Inactivated Infectious Bursal Disease Virus (IBDV), provided as an inactivated virus, and/or as an IBDV immunogenic protein or portion thereof, such as a recombinant or naturally occurring immunogenic protein or subunit(s) thereof. A typical IBDV antigen concentration is 3-60 μg/dose;

Avian Influenza Virus (AI), provided as an inactivated virus, and/or as an AI immunogenic protein or portion thereof, such as a recombinant or naturally occurring HA protein, or subunit(s) thereof. A typical AI antigen concentration is 10^{8.4} EID₅₀/dose (50% egg infective dose), or stated alternatively, 5-100 μg/dose; Infectious Bronchitis Virus (IBV), provided as an inactivated virus, and/or as an IBV immunogenic protein or portion thereof, such as a recombinant or naturally occurring spike protein, or subunit(s) thereof. A typical IBV antigen concentration is 10⁷ EID₅₀/dose;

Avian Reo Virus (ARV), provided as an inactivated virus, and/or as an ARV immunogenic protein or portion thereof, such as a recombinant or naturally occurring Sigma C protein, or subunit(s) thereof. A typical ARV antigen concentration is 10⁷ CCID₅₀/dose, or stated alternatively, 5-100 μg/dose;

Avian Metapneumovirus (AMPV), provided as an inactivated virus, and/or as an AMPV immunogenic protein or portion thereof, such as a recombinant or naturally occurring immunogenic protein or subunit(s) thereof. A typical AMPV antigen concentration is 10⁷ CCID₅₀/dose;

Salmonella bacterial infections, such as infections of Salmonella typhimurium, resulting in diseases such as Salmonella enteritis. Inactivated Salmonella bacteria is typically used in vaccines, but vaccines may also comprise a Salmonella immunogenic protein or portion thereof, such as a recombinant or naturally occurring immunogenic protein or subunit(s) thereof. A typical Salmonella antigen concentration is 10⁸ CFUs/dose (Colony Forming Units/dose);

Infectious coryza, resulting from Avibacterium paragallinarum, provided as an inactivated bacteria, and/or as a Avibacterium paragallinarum immunogenic protein or portion thereof, such as a recombinant or naturally occurring immunogenic protein or subunit(s) thereof. A typical Avibacterium paragallinarum concentration is 10⁸ CFUs/dose;

Colibacillosis, resulting from Escherichia coli infection. E. coli typically is provided as an inactivated bacteria, and/or as a E. coli immunogenic protein or portion thereof, such as a recombinant or naturally occurring immunogenic protein or subunit(s) thereof. A typical E. coli concentration is 10⁸ CFUs/dose;

Fowl cholera, resulting from Pasteurella multocida infection. P. multocida typically is provided as an inactivated bacteria, and/or as a P. multocida immunogenic protein or portion thereof, such as a recombinant or naturally occurring immunogenic protein or subunit(s) thereof. A typical P. multocida concentration is 10⁸ CFUs/dose;

Aquaculture is the rearing of aquatic species in agricultural settings, and is the fastest growing food producing sector globally. Based on FAO reports, aquaculture has surpassed production of beef and poultry, and accounts for one third of global food production. Intensification of production systems, the increase in species introduced to aquaculture and the environmental changes affecting water (e.g. salinity, pH, CO₂) have resulted in emerging infectious diseases, which pose a tremendous challenge for the expansion of aquaculture.

Disease prevention is a multifaceted strategy, in which vaccination is of upmost importance. Fish vaccination is environmentally friendly, has significantly reduced the use of antibiotics in aquaculture and has helped to control some diseases. Inactivated vaccines account for the largest proportion of vaccines used in aquaculture, yet their level of protection is equivocal. Whether administered by injection, immersion or co-administered with feed, novel formulations, allowing for better antigen stability and longer duration of immunity, are required for both bacterial and viral pathogens that afflict fish.

Inactivated/attenuated or recombinant antigens, generated as single or multi valent (listed but not limited to those of Table 1) preparations, are administered in PGPR formulations according to the present invention to fish individually orally, such as through their feed or by forced oral administration, or by injection, such as intramuscularly or intraperitoneally. In alternative embodiments, PGPR vaccine formulations according to the present invention can be administered simultaneously to an entire fish population contained in a body of water by spraying, dissolving and/or immersing the fish in water comprising the vaccine. Such population vaccination methods can be used in various environments such as ponds, aquariums, natural habitat, fish farms and fresh-water reservoirs.

TABLE 2
A Non Exhaustive List of Pathogens Afflicting Fish

| Disease | Pathogen | Major Fish Host |
|---|---|---|
| Viral nervous necrosis | NNV (Noda virus) | Sea bass, Sea bream, |
| | | Grouper sp, Tilapia, |
| | | Asian sea bass |
| Infectious hematopoietic | IHNV | Salmonids |
| necrosis | (Rhabdovirus) | |
| Infectious salmon anemia | ISAV | Atlantic Salmon |
| | (Orhtomyxovirus) | |
| Infectious pancreatic | IPNV | Salmonids, sea bass, |
| necrosis | (Birnavirus) | sea bream, turbot, cod |
| Pancreatic disease virus | SAV (alphavirus) | Salmonids |
| Infectious spleen and | ISKNV or RSIV | Sea bass, grouper, |
| kidney necrosis | (Iridovirus) | tilapia, sea bream |
| Tilapia Lake virus disease | TiLV | Tilapia |
| Enteric redmouth disease | Yersinia ruckeri | Salmonids |
| Vibriosis | Vibrio sp | Salmonids, groupers, |
| | (anguillarum, | sea bass, sea bream, |
| | ordalii, | yellow tail, cod, |
| | salmonicida) | halibut |
| Furunculosis | Aeromonas | Salmonids |
| | salmonicida sp | |
| Bacterial kidney disease | Renibacterium | Salmonids |
| | salmoninarum | |
| Enteric septicemia of | Edwarsiella | Catfish |
| catfish | ictalurid | |
| Columnaris disease | Flavobacterium | Tilapia, Sea bream, |
| | columnaris | sea bass, turbot, |
| | | salmonids, catfish |
| Pasteurellosis | Photobacterium | Sea bass, sea bream |
| | damselae | |
| | piscicida | |
| Lactococciosis | Lactococcus | Trout, amberjack, |
| | garviae | yellowtail |
| Streptococcus infections | Streptococcus sp. | Tilapia, sea bass, |
| | | sea bream, trout, |
| | | yellow tail |
| Salmonid rickettsial | Piscirickettsia | Salmonids |
| septicemia | salmonis | |
| Motile Aeromonas | Aeromonas sp. | Catfish |
| septicemia | | |
| Wound disease | Moritella viscosa | Salmonids |
| Tenacibaculosis | Tenacibaculum | Salmonids, Turbot |
| | maritimum | |

Specific fish disease and immunogenic components for treating such diseases include the following:

Streptococcus iniae, typically used in vaccines as inactivated bacteria, and/or as an immunogenic protein or portion produced by S. iniae, such as a recombinant or naturally occurring immunogenic protein or subunit(s) thereof. A typical S. iniae concentration in vaccine formulations is 0.01-0.04 OD/dose (optical density/dose).

Photobacterium, typically used in vaccines as inactivated bacteria, and/or as a immunogenic protein or portion of an immunogenic protein of Photobacterium, such as a recombinant or naturally occurring immunogenic protein or subunit(s) thereof. A typical Photobacterium concentration is 0.01-0.04 OD/dose (optical density).

Vibrio bacteria, typically used in vaccines as inactivated bacteria, and/or as a immunogenic protein or immunogenic portion of a Vibrio protein, such as a recombinant or naturally occurring immunogenic protein or subunit(s) thereof. A typical Vibrio concentration is 0.01-0.04 OD/dose (optical density).

### C. Oils and Surfactants

Disclosed vaccine embodiments may be formulated using PGPR without added surfactant or surfactants, such as by combining PGPR with a base water-in-oil composition. These PGPR formulations do also include an immunogenic component, such as an inactivated virus, an inactivated bacteria, or both, and/or an immunogenic bacterial and/or viral protein or portion thereof, such as a recombinant or naturally occurring immunogenic protein or subunit(s), as discussed above.

Any oil known by a person of ordinary skill in the art, or oil hereafter developed, that is suitable for vaccine formulation can be used to form PGPR formulations according to the present invention. Suitable oils include mineral oil, vegetable oil, and combinations thereof. Mineral oil has been used for certain exemplary embodiments of the present invention, such as a C₁₅-C₃₀ mineral oil.

Disclosed emulsion compositions, such as water-in-oil emulsions having various relative amounts of water and oil, such as 20% water to 50% water and 80% oil to 50% oil, indicated herein as a 20-50:80-50 water-in-oil emulsion, more typically a 20-30:80-70 water-in-oil emulsion, also may include at least one, and potentially plural, surfactants, such as a first surfactant and a second surfactant. Any surfactant known by a person of ordinary skill in the art, or surfactant hereafter developed, that is suitable for vaccine formulation can be used to form PGPR formulations according to the present invention. Suitable surfactants include, by way of example and without limitation: Sorbitol (S and T), Polyethylene-polypropylene glycol (F-68), Polysorbate 20 (PS20), polysorbate 40 (PS40), polysorbate 60 (PS60), polysorbate 80 (PS80), mannide monooleic acid, ethoxylated derivatives of oleic acid mannitan ester, decaglyceryl monolaurate, glyceryl monostearate, 2,6,10,15,19,23-hexamethyltetracosane (Squalane), polyethoxylated fatty acid (e.g., stearic acid), (SIMULSOL® M-53), polyethoxylated isooctylphenol/formaldehyde polymer, (TYLOXAPOL®), polyoxyethylene fatty alcohol ethers (BRIJ®), polyoxyethylene nonylphenyl ethers (TRITON® N), polyoxyethylene isooctylphenyl ethers (TRITON® X), copolymers of ethylene oxide (EO), propylene oxide (PO), and/or butylene oxide (BO) (DOWFAX™) phosphatidylcholine (lecithin), nonylphenol ethoxylates (Tergitol™ NP series), polyoxyethylene fatty ethers derived from lauryl, cetyl, stearyl and oleyl alcohols (Brij surfactants). Certain disclosed exemplary embodiments of the present invention used the following surfactants:

sorbitans, typically synthetic, non-ionic compounds, and typically having a formula weight of about 600 g/mol to 1,200 g/mol;

sorbitan oleates, such as sorbitan monoleate, shown below,

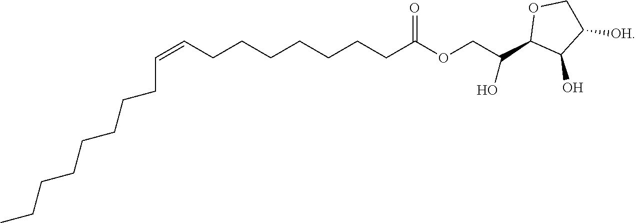 [SMILES: CCCCCCCC/C=C\CCCCCCCC(=O)OCC(O)[C@H]1OC[C@H](O)[C@H]1O]

polysorbates, such as polysorbate 80, shown below,

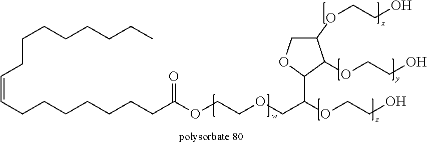

Block polymers, having a formula weight (FW) of about 4,000 g/mol;

Mannide derivatives, typically synthetic compounds having a FW of 750 g/mol;

Ddab surfactants, typically having a cationic polar head, and a FW of 630 g/mol;

Saponins, naturally occurring and typically having a non-ionic polar head, and a FW of 2,000 g/mol;

Phospholipids having an amphoteric polar head, and a FW of about 900 g/mol;

Lipopolysaccharides, having a FW of about 4,000 g/mol; and combinations of such surfactants.

Certain disclosed embodiments comprise: a base water-in-oil (30:70) emulsion; 0.05% to 2.0% PGPR; an immunogenic component; a first surfactant, such as 0.25% to 2.5%, typically about 1%, of a first surfactant, such as polysorbate 80; and a second surfactant, such as 2% to 6% of a second surfactant, typically about 3% of a second surfactant, such as a sorbitan oleate, such as sorbitan monooleate.

### D. PGPR-Based Vaccine Formulation Vaccination Results

Initial trials using PGPR vaccine formulations according to the present invention demonstrated that the addition of PGPR to standard W/O poultry vaccines improved the quality of the emulsion, prevented phase separation and improved stability. The effect of PGPR addition to the oil phase on the emulsion characteristics was dose dependent. A range of 0.06 to 0.5% PGPR was tested initially and found to improve the emulsion stability.

However, it was clear that there was a negative correlation between the PGPR concentration and vaccine efficacy for certain embodiments when tested 3 weeks after immunization. As the antigen concentration did not change in the vaccine, it currently is believed that PGPR stabilized the vaccine emulsion in vivo, thereby delaying antigen release and presentation of the antigen to the immune system.

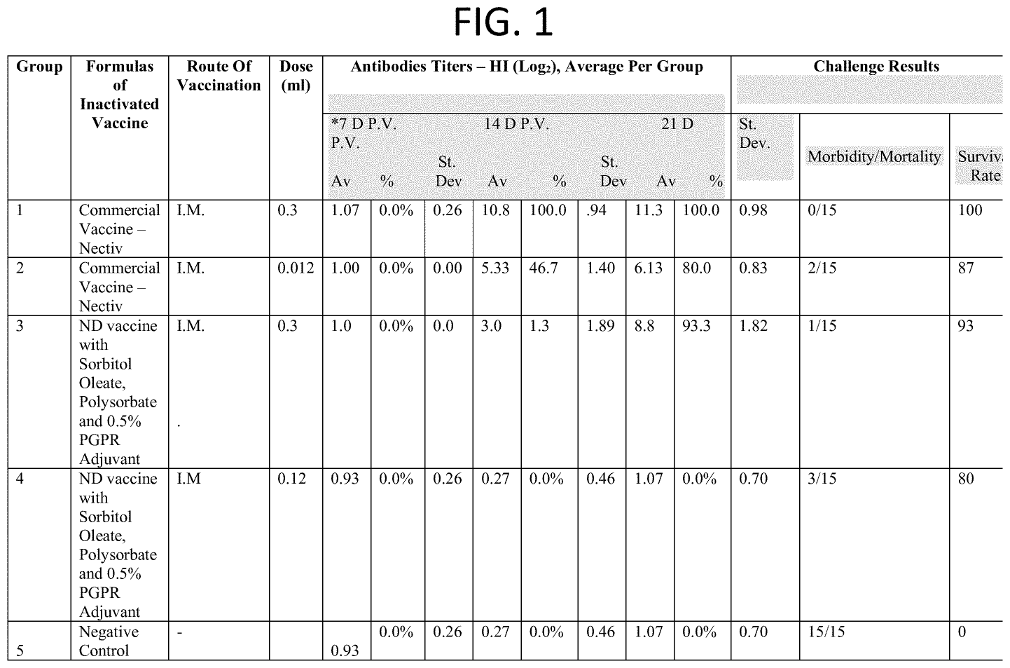

More recent studies demonstrated that by adding PGPR to a vaccine emulsion, with certain embodiments using 0.5% PGPR, the immune response in a subject was delayed by at least 7 days, and more typically at least 21 days and up to 34 days, following administration of an antigenic composition comprising PGPR. See FIG. 1. Adding PGPR to a vaccine emulsion therefore allows one to control the timing of the antigen release in vivo.

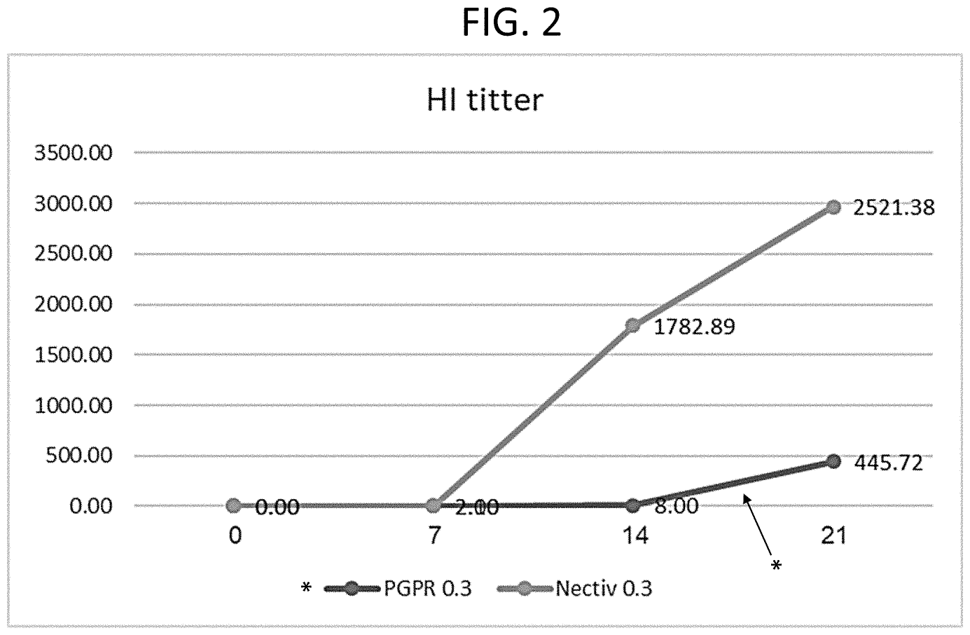

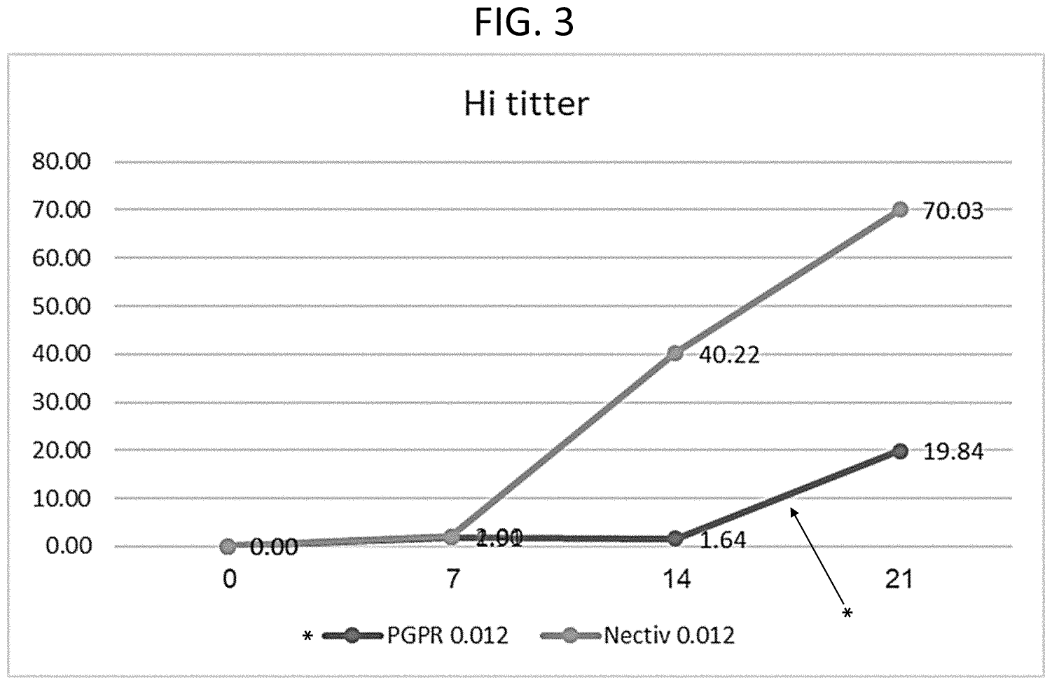

FIGS. 2 and 3 provide antibody titer results for vaccine formulations according to the present invention that include PGPR. More specifically, a vaccine formulation was made comprising inactivated Newcastle disease antigens, Sorbitan oleate and PGPR. These formulations were then administered subcutaneously to poultry at two different doses, an 0.3 ml dose and an 0.012 ml dose. FIGS. 2 and 3 show the immunity onset delay for vaccine formulations according to the present invention relative to a commercial vaccine, Nectiv, which is a W/O emulsion vaccine for Newcastle Disease provided by Phibro Animal Health Corporation. FIGS. 2 and 3 establish that

PGPR vaccine formulations according to the present invention can delay immunity onset for at least as much as 21 days subsequent to administration. As a result, the disclosed PGPR formulations allow vaccinators to control antigen release timing, which further enables optimizing the immune response under field conditions.

### IV. VACCINATION OF MATERNALLY IMMUNE YOUNG ANIMALS WITH PGPR FORMULATIONS

Breeding animals are immunized for two main purposes: first, to protect the animals during their growth period and reproductive period; and second, to confer protection to offspring through maternal immunization (i.e. passive transfer of IgG/IgY and IgA). The maternal protection may last weeks (chickens, dogs, cats) to months (cattle, sheep, pigs). However, while maternal immunity protects the animals, it also blocks the development of an acquired immune response by immunization with live or inactivated vaccines. In the specific case of poultry, maternal immunity against Newcastle disease virus (NDV), infectious bursal disease virus (IBDV), and Avian Influenza virus significantly decreases the efficacy of vaccinating day-old chicks in the hatchery.

PGPR formulations according to the present invention release inactivated virus or bacteria, or an antigen or antigens thereof, when the maternally-derived antibody level has decreased to a level that does not affect the development of acquired immune response. This typically occurs around the age of 9-14 days for chicks. As a result, disclosed PGPR vaccine formulations eliminate the need for a field injection, a common practice in areas with disease pressure. Field injections are administered to chicks once an effective decrease in the maternally-derived antibodies occurs. Disclosed PGPR vaccine formulations allow a producer to administer appropriate vaccines to a day-old chick, and thereby send a fully vaccinated chick to the field from the hatchery, and thereby avoid the necessity for a subsequent field injection.

### V. BOOSTING AND PRIMING EFFECT USING THE PGPR FORMULATION

Immunological memory, defined as the capacity of the immune system to respond more vigorously to a second contact with a given antigen than to the first contact, is the basis of the persistent protection afforded by the resolution of some infections and is the goal of vaccination. Memory is a system-level property of the immune system, which arises from an increase in the frequency of antigen-specific B and T cells, as well as from the differentiation of antigen-specific lymphocytes into memory cells, which can respond faster to antigens and to self-renew. The secondary immune response to an antigen is characterized by a shift to production of highly specific IgG (IgY) by activated plasma cells.

Recently the industry has struggled to reduce the number of immunization and injections administered to farm animals. Vaccinating production animal requires producers to handling and restrain each individual animal, a process which is considered stressful and painful to the animal. The presence of field vaccination teams also creates a risk of introducing pathogens to the farm.

Disclosed PGPR formulation embodiments provide producers the option to use the PGPR vaccine in combination and at substantially the same time as a standard vaccine (fast release). The standard vaccine induces a priming effect, and the PGPR formulation induces a boost effect a few weeks later. Each of the two separate vaccines can be administered separately and in any order.

A second option is to mix disclosed PGPR emulsion vaccine formulations together with a standard vaccine (fast release) to form a combined composition. This combining can be either at the formulation stage or just prior to injection to the animals. These mixed emulsion formulations may comprise 2 different micelles, a first based on PGPR, and a second based on standard micelle-forming components, such as polysorbate emulsions. Disclosed PGPR formulations reduce the number of vaccine injections without compromising either the initial immune response or the boost effect.

### VI. PHARMACEUTICAL FORMULATIONS COMPRISING PGPR

The PGPR formulations of the present invention may be combined with other formulation components commonly used in the field. Inactivated viruses or bacteria, and/or viral and/or bacterial antigens, can be formulated as pharmaceutical compositions and administered to a subject, such as a human or veterinary subject. Disclosed vaccine formulations may be administered by any of various known routes, as will be understood by a person of ordinary skill in the art, such as subcutaneously, intramuscularly, mucosally, intraperitoneally by injection, infusion, spraying, etc., and most typically subcutaneously or intramuscularly. Emulsions can be prepared using sterile aqueous solutions, dispersions, or emulsions comprising antigens, such as inactivated viruses or viral antigens. The ultimate dosage form typically is a sterile fluid that is stable under the conditions of manufacture and storage. Disclosed compositions may include preservatives to control microorganisms. Suitable preservatives include, by way of example and without limitation, parabens, chlorobutanol, phenol, sorbic acid, thiomersal, gentamycin, penicillin, and the like, and any and all combinations thereof. In many cases, it will be preferable to include isotonic agents, for example, sugars, such as sorbitol, including Sorbitan oleate and Polysorbate 80, buffers, or sodium chloride. Absorption of the injectable compositions can be additionally delayed, relative to using PGPR solely, by using for example, aluminum monostearate and/or gelatin.

Inactivated bacterial and/or viral vaccines, or vaccines comprising bacterial and/or viral antigens, may be systemically administered as a single dose. Alternatively, divided vaccine doses may be administered at appropriate time intervals over an administration period as will be determined for each individual subject. As yet another example, vaccines may be administered as two, three, four or more sub-doses per unit time, such as day, week, or month.

### VII. EXAMPLES

The following examples are provided to illustrate certain features of exemplary embodiments according to the present invention. A person of ordinary skill in the art will appreciate that the scope of the present invention is not limited to the particular features discussed by these examples.

### Example 1

This example evaluates delayed antigen release in influenza vaccines comprising PGPR at different concentrations administered to day-old commercial broiler chicks.

### A. Background

Avian influenza disease caused by the H9N2 influenza virus has a substantial economic impact on the poultry industry, although the disease is substantially controlled using commercially-available inactivated vaccine formulations. In Israel, for example, three monovalent H9N2 vaccines are routinely used to prevent the disease in broilers, egg layers and breeders.

One disclosed embodiment of the present invention concerns a new inactivated H9N2 vaccine for administration to day-old chicks. This new PGPR-based vaccine can be combined with other inactivated viruses or viral antigens, such as inactivated IBDV and NDV antigens. These new PGPR formulations are designed to bypass the neutralization effect provided by maternally-derived antibodies (MDA). PGPR delays antigen release and enables antigen activity after the level of maternal antibodies in the blood decreases. Accordingly, disclosed PGPR vaccine formulation embodiments of the present invention can be used to provide both delayed and slow antigen release.

The trial objective was to evaluate the immune response of broilers, with very high MDA, after vaccination with a H9N2 PGPR-based vaccine formulation at one day of age. The tested

H9N2 inactivated vaccines were based on the H9N2-215 strain and formulated in several concentrations of PGPR.

### B. Materials and Methods

### 1. Animals:

135-day old broiler chicks that were offspring to breeding flock 220 (Ross 40 weeks old) were held in floor pens in an experimental poultry house.

2. Vaccines: 4 batches of H9N2-215 influenza vaccine emulsions comprising different PGPR concentrations were prepared as illustrated below by Table 2.

### Emulsion—Aqueous Phase (21%):

a. 20m1 (20%) H9N2 inactivated antigen; and
b. 1 ml (1%) Polysorbate 80 (Sorbitol T)

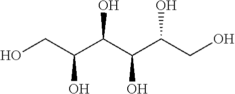 [SMILES: OC[C@@H](O)[C@@H](O)[C@H](O)[C@@H](O)CO]

(MONTANOX, which comprises polyethoxylated sorbitan esters).

To prepare an aqueous phase the mixture was stirred mechanically for a few minutes and then incubated at 37° C. and vortexed approximately every 10 minutes until the Polysorbate 80 dissolved.

### Emulsion—Oil Phase (79%):

a. Mineral oil-74.75-75.9 ml;
b. Sorbitan oleate (Sorbitol S) -3 ml (3%); and
c. PGP , 0.1-1.25 ml.

### 3. Emulsion Preparation:

The oil phase was mixed in a 250 ml glass container for 30 seconds using a Silverson homogenizer. The aqueous phase was slowly added to the oil phase while stirring at a speed of 6,000 RPM for one minute and 45 seconds with the Silverson's speed set to 8,000 rpm. HDPE bottles were filled with the resulting emulsion.

TABLE 3
Formulation Compositions

| | | Poly- | | | | |
|---|---|---|---|---|---|---|
| | | sorbate | | Sorbitan | | Volume |
| Batch # | Antigen % | 80% | Oil % | oleate % | PGPR % | (ml) |
| M-1-2470 | 20 | 1 | 75.9 | 3 | 0.1 | 100 |
| M-1-2471 | 20 | 1 | 75.5 | 3 | 0.5 | 100 |
| M-1-2472 | 20 | 1 | 75.25 | 3 | 0.75 | 100 |
| M-1-2473 | 20 | 1 | 74.75 | 3 | 1.25 | 100 |

*Stated percents are by volume

### 4. Commercial Avian Influenza Vaccines:

215, Batch No. 24011040;

947, Batch No. 24021037

1052, Batch No. 24031046

### C. Trial Design:

The chicks were randomly divided into 9 groups. Each group contained 15 birds.

Groups 1+3+4+5+6+7 were vaccinated 0.2 ml /dose at one day of age by SC Injection.

Groups 8+9 were vaccinated 0.3 ml /dose on day 13 of age by SC application.

### D. Experimental Groups:

Experimental groups are described below in Table No. 4.

TABLE 4

| | | Vaccination | Vaccination |
|---|---|---|---|
| Group | Vaccine | Age (Day) | Dose (ml) |
| 1 | Commercial AI 215 strain | 1 | 0.2 |
| 2 | Unvaccinated control | 1 | 0.2 |
| 3 | Mixture of commercial | 1 | 0.2 |
| | vaccines (strains 215, 947 and | | |
| | 1052)* | | |
| 4 | PGPR 0.1% AI 215 strain | 1 | 0.2 |
| 5 | PGPR 0.5% AI 215 strain | 1 | 0.2 |
| 6 | PGPR 0.75% AI 215 strain | 1 | 0.2 |
| 7 | PGPR 1.25% AI 215 strain | 1 | 0.2 |
| 8 | Commercial vaccine of strain | 13 | 0.3 |
| | 215 | | |
| 9 | Mixture of commercial | 13 | 0.3 |
| | vaccines (strains 947, | | |
| | 215, 1052)* | | |

*Mixture of commercial vaccines that was prepared before injection by mixing equal volumes of three vaccines after warming the vaccines within the test tube.
** the antigenic load per dose did not change between the groups, all included 20% of allantoic fluid that contains the antigen

### E. Evaluated Parameters:

Chickens were bled on days 1, 7, 13, 21, 28 and 35, and blood was tested for antibody response using a hemagglutination inhibition (HI) test and 8HA units of the 215 antigen.

### F. Results

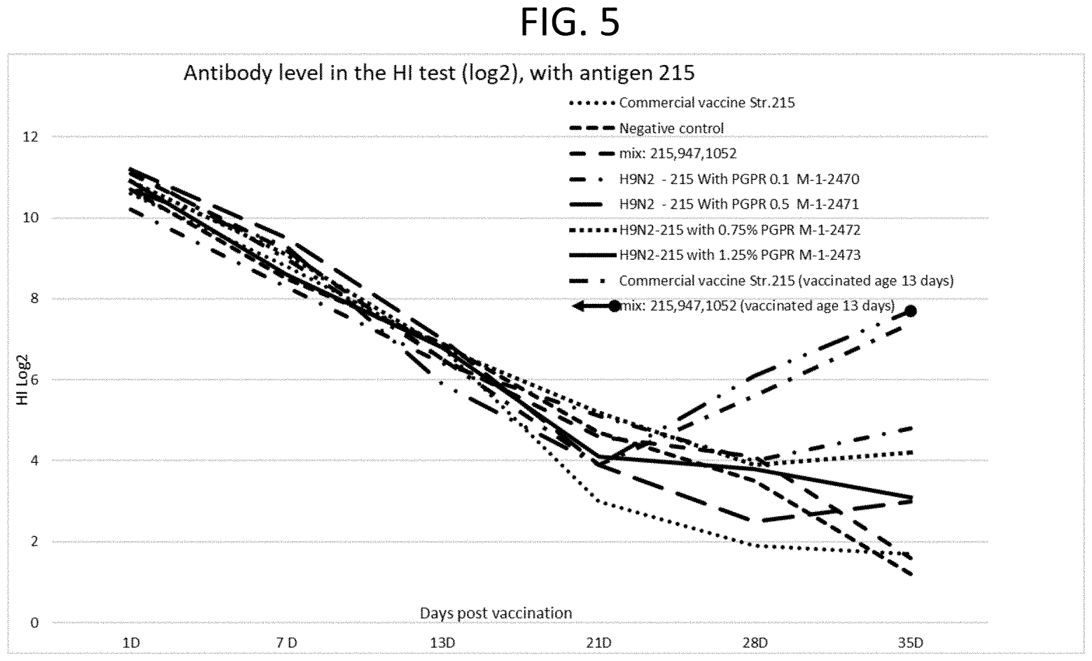

Serological results are presented in Tables 5 and 6, and FIG. 5.

TABLE 5
Antibody Titer in the HI Test (Log2), with Antigen 215

| | | Vaccination | Vaccination | | | | | | |
|---|---|---|---|---|---|---|---|---|---|
| Group | Vaccine | age (day) | dose (ml) | 1 D | 7 D | 13 D | 21 D | 28 D | 35 D |
| 1 | Commercial | 1 | 0.2 | 10.6 | 8.8 | 6.8 | 3 | 1.9 | 1.7 |
| | A1 215 strain | | | | | | | | |
| 2 | Unvaccinated | 1 | 0.2 | 10.7 | 8.5 | 6.9 | 4.7 | 3.5 | 1.2 |
| | controlled | | | | | | | | |
| 3 | Mixture of | 1 | 0.2 | 11.1 | 9.2 | 6.5 | 4.6 | 4.1 | 1.6 |
| | commercial | | | | | | | | |
| | vaccines | | | | | | | | |
| | (strains 215, | | | | | | | | |
| | 947 and | | | | | | | | |
| | 1052) | | | | | | | | |
| 4 | PGPR 0.1% | 1 | 0.2 | 10.2 | 8.3 | 6.4 | 5.1 | 4 | 4.8 |
| | A1 215 strain | | | | | | | | |
| 5 | PGPR 0.5% | 1 | 0.2 | 11.2 | 9.5 | 7 | 3.9 | 2.5 | 3 |
| | A1 215 strain | | | | | | | | |
| 6 | PGPR 0.75% | 1 | 0.2 | 10.9 | 9.1 | 6.8 | 5.2 | 3.9 | 4.2 |
| | A1 215 strain | | | | | | | | |
| 7 | PGRP 1.25% | 1 | 0.2 | 10.9 | 8.6 | 6.8 | 4.1 | 3.8 | 3.1 |
| | A1 215 strain | | | | | | | | |
| 8 | Commercial | 13 | 0.3 | 11.2 | 9 | 6.5 | 3.9 | 5.6 | 7.7 |
| | vaccine of | | | | | | | | |
| | strain 215 | | | | | | | | |
| 9 | Mixture of | 13 | 0.3 | 10.7 | 9.3 | 5.9 | 3.9 | 6.1 | 7.7 |
| | commercial | | | | | | | | |
| | vaccines, | | | | | | | | |
| | (strains 947, | | | | | | | | |
| | 215, 1052) | | | | | | | | |

TABLE 6
Percentage of Birds with HI Titer
Higher than 3 Units (Antigen 215)

| | | | | | % Chickens |
|---|---|---|---|---|---|
| | | Vaccina- | Vaccina- | | with Titer |
| | | tion | tion | HI Titer | Over 3 |
| Group | Vaccine | Age (Day) | Dose (Ml) | at 35D | (Log2) |
| 1 | Commercial | 1 | 0.2 | 1.7 | 14 |
| | A1 215 strain | | | | |
| 2 | Unvaccinated | 1 | 0.2 | 1.2 | 13 |
| | controlled | | | | |
| 3 | Mixture of | 1 | 0.2 | 1.6 | 14 |
| | commercial | | | | |
| | vaccines | | | | |
| | (strains 215, | | | | |
| | 947 and 1052) | | | | |
| 4 | PGPR 0.1% | 1 | 0.2 | 4.8 | 67 |
| | A1 215 strain | | | | |
| 5 | PGPR 0.5% | 1 | 0.2 | 3 | 40 |
| | 215 strain | | | | |
| 6 | PGPR 0.75% | 1 | 0.2 | 4.2 | 57 |
| | A1 215 strain | | | | |
| 7 | PGPR 1.25% | 1 | 0.2 | 3.1 | 36 |
| | A1 215 strain | | | | |
| 8 | Commercial | 13 | 0.3 | 7.4 | 93 |
| | vaccines of | | | | |
| | strain 215 | | | | |
| 9 | Mixture of | 13 | 0.3 | 7.7 | 100 |
| | commercial | | | | |
| | vaccines, | | | | |
| | (strains 947, | | | | |
| | 215, 1052) | | | | |

The H9 antibody level was evaluated in all chicks once a week starting at one day of age until 35 days of age. All broiler chicks had very high maternal antibody titer at one day of age (higher than expected for day old chicks). The maternal antibody titer declined by approximately 2 logs (base 2) every week (T_{1/2} of 3.5 days). At 35 days of age the HI titer of the negative control group (group 2) was 1.2.

The commercial vaccines groups (groups 1 and 3) that were injected at one day of age were not able to induce any increase in antibody titer and demonstrated antibody titer decline that was equivalent to the negative control group (group 2).

The commercial vaccine of strain 215, and the mixture of the 3 commercial vaccines (groups 8 and 9), injected at 13 days of age when MDA titer declined to HI 6 induced significant antibody increases starting at 28 days of age and maintained titers higher than HI 3.9 through all the observation period.

For groups 4, 6, and 7 (0.1%, 0.75%, 1.25% PGPR experimental vaccines, respectively) up to the age of 28 days there was a decrease in antibody titer similar to a negative control group. Groups 4 and 6 (0.1%, 0.75%, PGPR) demonstrated significant titer increase at 35 days of age to HI of 4.9 and 4.2, respectively, in comparison to HI of 1.2 in the control group (group 2). About 60% of the birds in groups 4 and 6 had HI titers higher than 3 at 35 days.

Group 5 and 7 (0.5%, 0.1.25%, PGPR) demonstrated lower titer increases at 35 days of age, with HI titers of 3and 3.1, respectively.

### G. Conclusions:

The PGPR-based formulations of the present invention induced HI titers higher than 3 at 28 and 35 days of age, and the 0.1 and 0.75% formulations induced titers higher than 4 in about 60% of the birds with an HI of 3.

The PGPR formulations were able, despite the maternal antibodies, to induce active production of antibodies against the inactivated H9N2 and a dose response effect was not demonstrated.

### Example 2

To demonstrate the efficacy of disclosed PGPR vaccine formulations for aquaculture, 100 fish will be divided into two groups of 50 fish each. After acclimatization, one group of fish will be vaccinated with a PGPR vaccine formulation according to the present invention via one of the methods described above. The control group of fish will be sham immunized. 3 to 4 weeks later fish will be challenged with virulent virus. Efficacy of PGPR vaccine formulations according to the present invention will be determined by the difference in the ratio of the fish mortality between both groups.

We claim:

1. A controlled release emulsion vaccine formulation comprising 0.015 to 6% polyglycerol polyricinoleate (PGPR).

2. The controlled release vaccine formulation according to claim 1 comprising 0.1% to 1.25% PGPR.

3. The controlled release vaccine formulation according to claim 1 comprising a 20-50:80-50 water-in-oil emulsion.

4. The controlled release vaccine formulation according to claim 1 further comprising a single surfactant sufficiently polar to create an emulsion.

5. The controlled release vaccine formulation according to claim 1 comprising 0.25% to 2.5% of a first surfactant, and 2% to 6% of a second surfactant.

6. The controlled release vaccine formulation according to claim 5, wherein the first surfactant is polysorbate 80 and the second surfactant is a sorbitan oleate.

7. The vaccine formulation according to claim 1, formulated for fowl and comprising:
inactivated Newcastle Disease Virus (NDV) and/or at least one NDV immunogenic protein or portion thereof;
inactivated Infectious Bursal Disease Virus (IBDV) and/or at least one IBDV immunogenic protein or portion thereof;
inactivated Avian Influenza Virus (AI) and/or at least one AI immunogenic protein or portion thereof;
inactivated Infectious Bronchitis Virus (IBV) and/or at least one IBV immunogenic protein or portion thereof;
inactivated Avian Reo Virus (ARV) and/or at least one ARV immunogenic protein or portion thereof; and/or
inactivated Avian Metapneumovirus (AMPV) and/or at least one AMPV immunogenic protein or portion thereof.

8. The vaccine formulation according to claim 7, comprising:
an NDV antigen concentration of about 50 PD₅₀/dose (50% protective dose), or 5 μg/dose-50 μg/dose;
an IBDV antigen concentration of 3 μg/dose-60 μg/dose;
an AI antigen concentration of about 10⁸⁴ EID₅₀/dose (50% egg infective dose), or 5 μg/dose-100 μg/dose;
an IBV antigen concentration;
an ARV antigen concentration of 10⁷ CCID₅₀/dose, or 5 μg/dose-100 μg/dose; or
an AMPV antigen concentration of 10⁷ CCID₅₀/dose.

9. The vaccine formulation according to claim 1, formulated for fowl and comprising:
inactivated Salmonella and/or an inactivated Salmonella immunogenic protein or portion thereof;
inactivated Avibacterium paragallinarum and/or an inactivated A. paragallinarum immunogenic protein or portion thereof;
inactivated Escherichia coli and/or an inactivated E. coli immunogenic protein or portion thereof; and/or
inactivated Pasteurella multocida and/or an inactivated immunogenic protein or portion thereof.

10. The vaccine formulation according to claim 9 and comprising 10⁸ CFUs/dose of each antigenic component.

11. The vaccine formulation according to claim 1, formulated for fish and comprising 0.01-0.04 OD/dose of:
inactivated Streptococcus iniae and/or an inactivated immunogenic S. iniae protein or portion thereof;
inactivated Photobacterium and/or an inactivated Photobacterium immunogenic protein or portion thereof; and/or
inactivated Vibrio and/or an immunogenic protein or immunogenic portion of a immunogenic Vibrio protein.

12. The vaccine according to claim 1, comprising an inactivated H9N2 PGPR emulsion-based vaccine for day-old chicks.

13. The vaccine according to claim 12, comprising:
21% of an aqueous emulsion phase comprising 20% H9N2 inactivated antigen, and 1 ml (1%) Polysorbate 80; and
79% of an oil emulsion phase comprising 76% mineral oil, 3 ml (3%) Sorbitan oleate, and 0.1-1.25% PGPR.

14. The vaccine according to claim 1 wherein an immune response is delayed in a subject receiving the vaccine by 7-21 days relative to a subject receiving a vaccine that is the same vaccine except that it does not include PGPR.

15. The vaccine according to claim 1 further comprising a second standard fast release vaccine that is administered as a combination or as a composition.

16. A controlled release, water-in-oil emulsion vaccine formulation, comprising:
a 20-50:80-50 water-in-oil emulsion;
0.015 to 6% polyglycerol polyricinoleate (PGPR);
optionally 0.25% to 2.5% of a first surfactant;
optionally 2% to 6% of a second surfactant; and
an immunogenic component selected from inactivated Newcastle Disease Virus (NDV) and/or at least one NDV immunogenic protein or portion; inactivated Infectious Bursal Disease Virus (IBDV) and/or at least one IBDV immunogenic protein or portion thereof; inactivated Avian Influenza Virus (AI) and/or at least one AI immunogenic protein or portion thereof; inactivated Infectious Bronchitis Virus (IBV) and/or at least one IBV immunogenic protein or portion thereof;
inactivated Avian Reo Virus (ARV) and/or at least one ARV immunogenic protein or portion thereof; inactivated Avian Metapneumovirus (AMPV) and/or at least one AMPV immunogenic protein or portion thereof; inactivated Salmonella and/or an inactivated Salmonella immunogenic protein or portion thereof; inactivated Avibacterium paragallinarum and/or an inactivated A. paragallinarum immunogenic protein or portion thereof; inactivated Escherichia coli and/or an inactivated E. coli immunogenic protein or portion thereof; inactivated Pasteurella multocida and/or an inactivated immunogenic protein or portion thereof; inactivated Streptococcus iniae and/or an inactivated immunogenic S. iniae protein or portion thereof; inactivated Photobacterium and/or an inactivated Photobacterium immunogenic protein or portion thereof; and/or inactivated Vibrio and/or an immunogenic protein or immunogenic portion of a immunogenic Vibrio protein.

17. The controlled release, water-in-oil emulsion vaccine formulation according to claim 16, comprising 1% Polysorbate 80 and 3% sorbitan monooleate.

18. A combination, comprising:
a first vaccine formulation comprising polyglycerol polyricinoleate (PGPR) according to claim 1; and
a second standard fast release vaccine.

19. The combination according to claim 18 wherein the vaccines are administered separately in any order or simultaneously.

20. The combination according to claim 18 wherein the combination is a composition comprising the first and second vaccines.

21. A method, comprising vaccinating a subject with a vaccine formulation according to claim 1, or a combination comprising a vaccine formulation according to claim 1.

22. The method according to claim 21 wherein PGPR-based formulations comprising inactivated H9N2 induced HI titers higher than 3 at 28 and 35 days of age.

23. The method according to claim 21 wherein the subject is poultry, and 0.1 and 0.75% PGPR formulations comprising inactivated H9N2 induced titers higher than 4 in about 60% of birds with an HI of 3.

24. The method according to claim 21 comprising administering a water-in-oil emulsion vaccine comprising PGPR to day-old chicks to induce active production of antibodies against inactivated H9N2 despite the presence of maternal antibodies.

25. The method according to claim 21 further comprising vaccinating the subject in combination with a standard vaccine that does not comprise PGPR, whereby the standard vaccine induces a priming effect, and the PGPR formulation induces a boost effect 21-35 days later.

26. The method according to claim 21 wherein the subject is poultry or an aquatic species.

27. The method according to claim 26 wherein the subject is a day-old chick or a fish species.

28. A method for making an inactivated emulsion vaccine comprising PGPR, the method comprising:
forming an aqueous phase comprising inactivated antigens;
forming an oil phase comprising PGPR; and
forming an emulsion comprising the aqueous phase and the oil phase.

29. The method according to claim 28, wherein:
the aqueous phase comprises H9N2 antigens and Polysorbate 80; and
the oil phase comprises mineral oil, a sorbitan oleate, and PGPR.

30. The method according to claim 28 comprising forming a composition comprising the first vaccine formulation and the standard vaccine formulation.
